(19) United States
(12) Patent Application Publication
(10) Pub. No.: US 2009/0227027 A1
(43) Pub. Date: Sep. 10, 2009

(54) COATED CELL CULTURE SURFACES AND METHODS THEREOF

(76) Inventors: Wendy A. Baker, Bath, NY (US); Theresa Chang, Painted Post, NY (US); Robert R. Hancock, JR., Corning, NY (US); Wanda J. Walczak, Big Flats, NY (US)

Correspondence Address: CORNING INCORPORATED, SP-TI-3-1, CORNING, NY, 14831, US

(21) Appl. No.: 12/390,726

(22) Filed: Feb. 23, 2009

Related U.S. Application Data
(60) Provisional application No. 61/068,578, filed on Mar. 7, 2008.

(51) Int. Cl. C12N 5/00

(52) U.S. Cl. 435/406; 435/404

(57) ABSTRACT

A composition for use, for example, in cell culture surface modification and in cell culture articles, as defined herein. Also disclosed are methods of making the compositions and articles, and methods of using the compositions and articles, as defined herein.

### CLAIMING BENEFIT OF PRIOR FILED U.S. APPLICATION

This application claims the benefit of U.S. Provisional Application Ser. No. 61/068,578, filed on Mar. 7, 2008. The content of this document and the entire disclosure of publications, patents, and patent documents mentioned herein are incorporated by reference.

### BACKGROUND

The disclosure is related to cell culture surfaces and methods of making and use of the surfaces.

### SUMMARY

The disclosure provides compositions for use, for example, in cell culture surface modification and to cell culture articles thereof. The disclosure also provides methods of making the compositions and articles. The disclosure further provides methods of using the compositions and articles.

### BRIEF DESCRIPTION OF THE DRAWINGS

FIG. 1 shows comparative results of albumin production of hepatocyte cells cultured on a substrate having different surface coat compositions, in embodiments of the disclosure.

FIGS. 2A to 2D show magnified (5× brightfield micrographs) images of a laurylmethacrylate-HEMA copolymer control coatings compared to exemplary coatings of a blend of laurylmethacrylate-HEMA copolymer and Methocel® that demonstrate enhanced cell attachment characteristics, in embodiments of the disclosure.

FIG. 3 is a magnified brightfield image of a portion of FIG. 2D, which indicates coated substrate surface topography or roughness, and cell attachment, in embodiments of the disclosure.

FIGS. 4A and 4B show examples of magnified images of locust bean gum coating delamination on a TCT substrate and a more robust, non-delaminating, coating of a blend of locust bean gum and Natrasol® 430, respectively, in embodiments of the disclosure.

FIGS. 5A to 5C show exemplary delamination response curves for several hydrogel-gum and hydrophobe-modified-polysaccharide blend combinations as a function of time in cell culture and % added hydrophobe modified cellulose, in embodiments of the disclosure.

FIGS. 6A to 6E show examples of magnified (20× micrographs) images of controls and several exemplary blended coatings of admixtures of hydrogel-gum (locust bean gum) and hydrophobe modified polysaccharide (Natrasol® 100), in embodiments of the disclosure.

FIG. 7 shows a chart of the % transmission vs distance as a measure of optical clarity as determined by a line scan in a cell-free area of blended materials and controls shown in FIG. 6, in embodiments of the disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and set forth only some of the many possible embodiments for the claimed invention.

### Definitions

“Attach,” “attachment,” “adhere,” “adhered,” “adherent,” “immobilized,” or like terms generally refer to immobilizing or fixing, for example, a cell, and like entities of the disclosure, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with a cell anchoring material, a compatibilizer, or a surface coating.

“Adherent cells” refers to a cell or a cell line or a cell system, such as a prokaryotic or eukaryotic cell, that remains associated with, immobilized on, or in certain contact with the outer surface of a substrate. Such type of cells after culturing can withstand or survive washing and medium exchanging process, a process that is prerequisite to many cell-based assays. “Weakly adherent cells” refers to a cell or a cell line or a cell system, such as a prokaryotic or eukaryotic cell, which weakly interacts, or associates or contacts with the surface of a substrate during cell culture. However, these types of cells, for example, human embryonic kidney (HEK) cells, tend to dissociate easily from the surface of a substrate by physically disturbing approaches such as washing or medium exchange. “Suspension cells” refers to a cell or a cell line that is preferably cultured in a medium wherein the cells do not attach or adhere to the surface of a substrate during the culture. However, these suspension cells can be attached to a surface through electrostatic interactions or covalent coupling. “Cell culture” or “cell culturing” refers to the process by which either prokaryotic or eukaryotic cells are grown under controlled conditions. “Cell culture” refers to the culturing of cells derived from multicellular eukaryotes, especially animal cells, and can also refer to the culturing of complex tissues, organs, pathogens, or like systems.

“Cell” or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including natural cells, genetically modified cells, synthetic cell constructs, cell model systems, and like artificial cellular systems. “Cell” can also include “pathogen” or like terms that refer to, for example, a virus, a bacterium, a prion, and like infectious entities, or combinations thereof.

“Cell system” or like term refers to a collection of more than one type of cells (or differentiated forms of a single type of cell), which interact with each other, thus performing a biological, physiological, or pathophysiological function. Such cell system includes an organ, a tissue, a stem cell, a differentiated hepatocyte cell, or like systems.

“Hydrogel” or like terms refer to a colloidal gel or like dispositions of matter in which water or an aqueous medium is the dispersed phase. A colloidal gel is a colloid in having a more solid form than a sol. Hydrogels are generally known, see for example, Merrill, et al., “Hydrogels for Blood Contact,” in N. A. Peppas, ed., Hydrogels in Medicine and Pharmacy, CRC Press, pp. 1-16 (1986), and Andrade, J. D., “Hydrogels for Medical and Related Applications: a symposium,” American Chemical Society. Division of Polymer Chemistry. p. 14-29, 1976. Hydrogels typically are amorphous, have considerable water content, such as from about 30 wt % or more, high surface wetting, and low surface energy characteristics. Additionally or alternatively, a hydrogel can be described as the formation of a colloid in which the disperse phase (colloid) has combined with the continuous phase (e.g., water) to produce a viscous jellylike product. Locust bean gum has been referred to as a hydrogel. The hydrophobic portion of hydrophobically modified cellulose is not water soluble. Hydroxyethylcellulose has been referred to as a hydrogel (see for example, Petrov et al., “Synthesis of biodegradable hydroxyethylcellulose cryogels by UV irradiation,” Polymer, Vol. 48, (17), 4943-4949, 2007).

“Bulk complex modulus” or like terms can be related to the elasticity or elastic modulus of the substrate coating or film measured dynamically under oscillatory strain. An ASTM method used to measure complex modulus is ASTM D 4440-07 “Standard Test Method for Plastics: Dynamic Mechanical Properties Melt Rheology,” where the complex modulus (CM) can be calculated from the formula:

CM=CV×F

where CM is complex modulus (G*, in Pa), CV is Complex Viscosity (Eta*, in Pa·s), and F is Frequency (omega, in rad/sec.).

“Include,” “includes,” or like terms means including but not limited to.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

“Consisting essentially of” in embodiments refers, for example, to a cell culture surface composition, a method of making or using a surface composition, formulation, or surface composition on the surface of a cell culture vessel or like surfaces, such as a biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface coating, a particular surface modifier or condition, or like structure, material, or process variable selected. Items that may materially affect the basic properties of the components or steps of the disclosure or that may impart undesirable characteristics to the present disclosure include, for example, aberrant affinity of a cell for the coated surface, anomalous or contrary cell activity of a cell or cell line for the coated surface or like activity, and like characteristics.

Thus, the claimed invention may suitably comprise, consist of, or consist essentially of: a cell culture composition having a hydrogel blend, cell culture articles having the hydrogel blend composition, a method of making the composition and articles, and a method of using the composition and articles, as defined herein.

The indefinite article “a” or “an” and its corresponding definite article “the” as used herein means at least one, or one or more, unless specified otherwise.

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, and like abbreviations).

Specific and preferred values disclosed for components, ingredients, additives, cell-types, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Cellulose is an compound of the formula (C₆H₁₀O₅)ₙ, a polysaccharide consisting of, for example, a linear chain of several hundred to over ten thousand linked glucose units.

Locust bean gum is a galactomannan polysaccharide of formula (I):

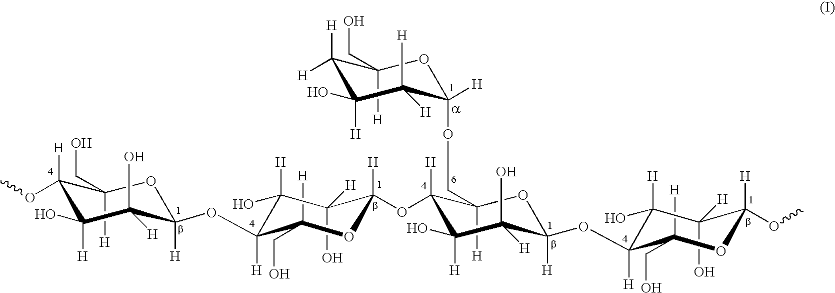

Locust bean gum (LBG) is a galactomannan polysaccharide consisting of mannopyranose backbone with branch points from their 6-positions linked to α-D-galactose residues. Locust bean gum has about 3.5 (for example, about 2.8 to about 4.9) mannose residues for every galactose residue (a mannose/galactose ratio of about 4). Galactomannan gums include, for example, locust bean gum (LBG), guar gum, cassia gum, tara gum, mesquite gum, and fenugreek gum. Guar gum is also a galactomannan polysaccharide consisting of a mannopyranose backbone. However, guar gum has more galactose branch points than locust bean gum. Guar gum has a mannose/galactose ratio of about 2. The mannose/galactose ratio is about 1:1 for mesquite gum and fenugreek gum, about 3:1 for tara gum and about 5:1 for Cassia gum. These structural difference cause guar gum to be more water soluble and to $ have a lower viscosity than locust bean gum. Locust bean gum is a galactomannan compound with lower galactose substitution and therefore it is less stiff or more flexible. The larger the mannose/galactose ratio, the less viscous and more water soluble the gum. Gums with a higher mannose/galactose ratio are less stiff or more flexible, while gums with a lower mannose/galactose ratio are more workable based on solubility, dispersion and emulsification properties. Locust bean gum has a higher bulk complex modulus (335-540 Pa) and is less elastic compared to cassia gum (5-13 Pa). Higher galactose substitution of these gums gives them improved solubility, dispensability, and emulsifability. Higher galactose substitution makes the galactomannan polysaccharides stiffer. Higher substitution provides the gums with lower viscosity and higher solubility.

Galactomannan gums, which are galactomannan polysaccharides, are gums which can be derived from natural sources, or from recombinant or synthetic sources. Galactomannan gums also include these gums which are purified or treated or modified by processes which may include, for example, enzymatic treatment, filtration, centrifugation, hydrolysis, freeze-thaw cycles, heating and chemical treatments or like modifications, mixtures of galactomannan gums with other galactomannan gums or non-galactomannan gums, and mixtures of galactomannan gums with other ingredients which optimize the cell culture characteristics.

Embodiments of the disclosure include gums which have been treated with alpha-galactosidase or other enzymes or chemical treatments, to “tune” the gums to provide the gum with desired characteristics as a coating for cell culture surfaces.

In embodiments of the disclosure, galactomannan gums, having mannose backbones and galactose side chains, in combination with other polymer components as defined herein, can provide a surface coating for cell culture which presents galactose moieties to cells in culture. For some cell types, these galactose moieties may provide a surface or growth bed which encourages the growth of cells in culture.

In embodiments, the disclosure can provide a 3-dimensional cell culture scaffold that can be obtained by, for example, selection of substrate, chemical functionalization of the polymers on the film surface, or a combination thereof, so that biologically relevant macromolecules or ligands can be attached. The functionalized polymers can be, for example, crosslinked by various methods to form the 3-D scaffolds.

Aqualon® is a high purity sodium carboxymethylcellulose (CMC) available from Hercules. Aqualon® is an anionic water-soluble polymer derived from cellulose that is physiologically inert and is an anionic polyelectrolyte that can be used as a starting material for preparing HMHEC compounds in embodiments of the disclosure.

Natrosol® hydroxyethylcelluloses (HEC), available from Hercules, are a family of nonionic water-soluble polymers derived from cellulose. Like Aqualon® cellulose gum (sodium carboxymethylcellulose), it is a cellulose ether, but it differs in that it is nonionic and its solutions are unaffected by cations. Natrosol® PLUS CS is an example of a commercially available hydrophobically modified HEC.

PolySurf 67® available from Hercules is a cetyl hydroxyethylcellulose of the formula:

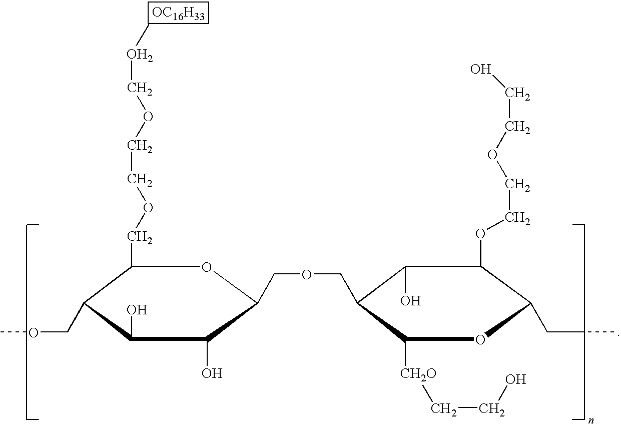

Methocel® cellulose ethers are water-soluble methylcellulose and water-soluble hydroxypropyl methylcellulose polymers available from Dow.

Cadherins are a class of type-1 transmembrane proteins. They play important roles in cell adhesion, ensuring that cells within tissues are bound together. They are dependent on calcium (Ca²⁺) ions to function. E-cadherin (epithelial) is a well characterized cadherin consisting of 5 cadherin repeats (C1˜EC5) in the extracellular domain, one transmembrane domain, and an intracellular domain that binds p120-catenin and beta-catenin. The intracellular domain contains a highly-phosphorylated region vital to beta-catenin binding and therefore to E-cadherin function. Beta-catenin can also bind to alpha-catenin. Alpha-catenin participates in regulation of actin-containing cytoskeletal filaments. In epithelial cells, E-cadherin-containing cell-to-cell junctions are often adjacent to actin-containing filaments of the cytoskeleton. E-Cadherins are a convenient target for analytical staining.

The disclosure relates to the use of blends of one or more modified cellulose material to modify cell culture substrates for use in, for example, liver cell culture. The cell culture substrates can be used in a variety of applications, such as ADMETox, cell harvesting, and like applications (see refs. 1, 2).

Matrigel® sandwich and the Collagen I sandwich have been cited as standards for hepatocyte culture as they maintain in vivo like function for extended periods (see ref. 3). However, since Matrigel® comes from a mouse tumor, it is not an adequate substrate for healthy human hepatocyte culture. Moreover, because both Matrigel® and collagen I are animal derived, they are subject to moderate to high lot-to-lot variability. Additionally, they can not be easily and reproducibly processed into a 3-D scaffold which has good stability and mechanical properties.

In embodiments the disclosure provides: a substrate or cell culture surface coating having a defined composition that is less complex than Matrigel®; a cell culture surface that maintains human liver cell function for an extended period, such as the time up to and inclusive of 14 days or other suitable culture times under suitable conditions; a cell culture surface that exhibits approximate in vivo-like cell culture that allows more predictive cell based assays; and a cell culture surface having convenient cell harvesting properties.

Certain water swellable polymers such as locust bean gum, are known to provide enhanced function for hepatocytes comparable to Matrigel®. However, such swellable polymers are unable to adhere satisfactorily to an underlying plastic substrate without mechanically forcing attachment Mechanically forced attachment to the substrate can decrease the amount of available cell growth area (see ref. 4).

In embodiments, the disclosure provides a cell culture composition comprising:

a hydrophobe modified polysaccharide comprising a polysaccharide having a plurality of hydrophobes, the hydrophobe having from 6 to about 22 carbon atoms, the hydrophobes comprise from about 1 to about 20 mole % and the polysaccharide comprises from about 80 to about 99 mole %; and

a water soluble, swellable hydrogel that is miscible with the hydrophobically modified cellulose.

The hydrophobe can be, for example, a C₁₂₋₂₀ alkyl and the polysaccharide comprises a cellulose. The hydrophobe can also be selected from the substituents, for example:

—O—R;

—S—R;

—O—C(═O)R;

—S—C(═O)R;

—S—C(═S)R;

—C(═O)R;

—O—CH₂—CH(OH)—CH₂—O—R;

—O—CH₂—CH(OH)—CH₂—O—C(═O)R; and

—O—CH₂—CH(OH)—CH₂—C(═O)R,

or combinations thereof, where R is a C₁₆₋₂₀ alkyl having a linear alkyl chain, a branched alkyl chain, or a mixture thereof, and the polysaccharide comprises a cellulose, an hydroxyalkyl cellulose, or mixtures thereof having from 0 to about 3 hydroxyalkyl groups for each saccharide of the polysaccharide, the hydroxyalkyl group having from 2 to 6 carbon atoms.

The hydrophobe modified polysaccharide can be, for example, a hydroxyethyl modified cellulose having from 0 to 3 hydroxyethyl groups for each glucose, the hydrophobe comprises a —O—C(═O)R, where R is C₁₆₋₂₀ alkyl, and having a total hydroxyethyl to hydrophobe mole ratio of from about 6:1 to about 1:1. The hydrophobe modified polysaccharide can be, for example, a hydroxyethyl modified cellulose having from 1 to 3 hydroxyethyl groups for each glucose, the hydrophobe comprises a —O—C(═O)R, where R is C₁₈ alkyl, and having, for example, from about 2 to about 15 mole % hydrophobe and from about 85 to about 98 mole % polysaccharide.

The cell culture compositions of the disclosure can include a hydrophobe modified cellulose that can have, for example, at least one of an alkylated hydroxyethyl cellulose, an alkylated cellulose, or like hydrophobe modified celluloses. The alkyl of the alkylated group can have, for example, from 6 to about 20 carbon atoms (C₆₋₂₀ alkyl), and the hydrophobically modified cellulose can have from about 0.5 wt % to about 20 wt % alkylation, such as a Polysurf 67®, Natrosol® Plus CS, Methocel®, and like hydrophobe modified celluloses. However, Ethocel,® for example, may be less desirable or unsatisfactory depending upon its combinations and concentrations because of its lower solubility in alcohol.

In embodiments, the cell culture composition can include, for example: from about 10 to about 90 wt % a hydrophobe modified cellulose; and from about 10 to about 90 wt % water soluble, swellable hydrogel. In embodiments, the cell culture compositions can also include, for example, from about 25 to about 75 wt % a hydrophobically modified cellulose; and from about 25 to about 75 wt % water soluble, swellable hydrogel.

The water soluble, swellable hydrogel can be, for example, at least one galactomannan gum selected from locust bean gum, carob bean bum, carob seed gum, carob gum, guar gum, cassia gum, tara gum, mesquite gum, fenugreek gum, or a combination thereof.

Although not bound by theory, it is believed that the hydrophobically modified polysaccharide, such as the hydrophobe modified cellulose (HMC), in combination with the hydrogel promotes adhesion of the hydrogel to the substrate, to provide for example, a more robust cell culture substrate coating. Alternatively or additionally, the hydrophobe modified polysaccharide in combination with the hydrogel provide viable cell culture coatings or films that are optically clear or substantially transparent. Use of the disclosed optically clear hydrophobe modified polysaccharide and hydrogel blend compositions can conveniently and advantageously obviate the of use stain when imaging cultured cells.

In embodiments, the disclosure provides a cell culture composition comprising:

a hydrophobe modified polysaccharide comprising a polysaccharide having a plurality of hydrophobes, the hydrophobe having from 6 to about 22 carbon atoms, the hydrophobes comprise from about 1 to about 20 mole % and the polysaccharide comprises from about 80 to about 99 mole %; and

a water swellable polymer that is immiscible with the hydrophobe modified polysaccharide, such as a copolymer of HEMA and alkylmethacrylate monomers.

The water swellable polymer can be, for example, of the formula:

—(CH₂—C(CH₃)(CO₂R¹))ₓ—(CH₂—C(CH₃)(CO₂—R²—OH))_{y}—

where

R¹ is a monovalent —C₁₀₋₁₄alkyl;

R² is a divalent —(C₂₋₆alkyl)-;

x is from about 10 to about 1,000; and

y is from about 10 to about 1,000.

The polymer of the formula can be, for example, R¹ is C₁₂H_{25,} and R² is —CH₂—CH₂—, also known as a copoly(laurylmethacrylate-2-hydroxyethylmethacrylate). In embodiments, the hydrophobe can be, for example, a C₁₂₋₂₀ alkyl and the polysaccharide can be, for example, a cellulose. The hydrophobe can be, for example, selected from the group structurally described above. Similarly, the polysaccharide can be, for example, as structurally described above.

In embodiments, the cell culture composition can be, for example: from about 5 to about 25 wt % a hydrophobe modified cellulose; and from about 75 to about 95 wt % a water swellable polymer. The water swellable polymer can be, for example, at least one of copolymer comprised of an hydroxyalkyl methacrylate, such as a 2-hydroxyethyl methacrylate (HEMA) monomer and an alkylmethacrylate, such as a laurylmethacrylate monomer. The water swellable polymer can be, for example, a copoly(laurylmethacrylate-2-hydroxyethylmethacrylate).

The hydrophobe modified polysaccharide in combination with the water swellable polymer can promote increased cell adhesion to a cell culture substrate coated with the combination, to provide, for example, a more robust cell culture substrate coating composition and the water swellable polymer imparts surface topography to the cell culture coated substrate.

In embodiments, the disclosure provides a cell culture article including, for example, the cell culture composition comprising: a hydrophobe modified polysaccharide comprising a polysaccharide having a plurality of hydrophobes, the hydrophobe having from 6 to about 22 carbon atoms, the hydrophobes comprise from about 1 to about 20 mole % and the polysaccharide comprises from about 80 to about 99 mole %; and a water soluble, swellable hydrogel that is miscible with the hydrophobically modified cellulose, coated on a suitable substrate.

In embodiments, the disclosure provides a cell culture article including, for example, the above mentioned composition of a hydrophobe modified polysaccharide comprising a polysaccharide having a plurality of hydrophobes, the hydrophobe having from 6 to about 22 carbon atoms, the hydrophobes comprise from about 1 to about 20 mole % and the polysaccharide comprises from about 80 to about 99 mole %; and a water swellable polymer that is immiscible with the hydrophobe modified polysaccharide, coated on a substrate.

In embodiments, the disclosure provides the articles having the abovementioned coating, the coating can further including, for example, a biologically active compound including, for example, at least one of an amino acid, a peptide, a polypeptide, a protein, a carbohydrate, a lipid, a polysaccharide, a nucleic acid, a nucleotide, a polynucleotide, a glycoprotein, a lipoprotein, a glycolipid, a glycosaminoglycan, a proteoglycan, a growth factor, a differentiation factor, a hormone, a neurotransmitter, a pheromone, a chalcone, a prostaglandin, an immunoglobin, a monokine, a cytokine, an humectant, a fibrous protein, an adhesion compound, a de-adhesion compound, an enzyme, or a combination thereof.

In embodiments the disclosure provides a method for making the disclosed compositions and articles comprising, for example, blending the components in at least one of a homogenous mixture, a layered structure, a multiple-layered structure, and like structures, or combination s thereof. The layered structure can be, for example, a cell encapsulating structure, a sandwich structure having a second layer of blended material atop the cells seeded on the coated surface, and like structures, or combinations thereof.

In embodiments the disclosure provides a method for cell culture comprising:

providing a substrate coated with one or more of the disclosed cell culture compositions;

contacting the coated substrate with a cell culture for a suitable or sufficient time to establish functional cells; and

harvesting the cells from the substrate.

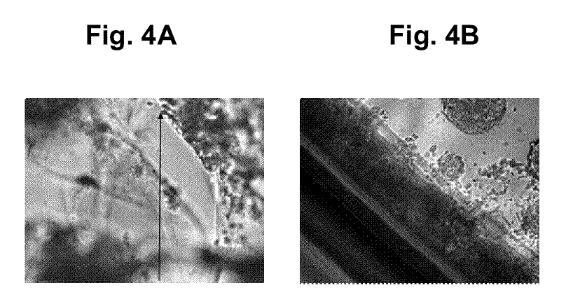

In embodiments the disclosure provides a substrate which enhances hepatocyte function, is reproducible and stable, is not animal derived, and provides good adherence to an underlying substrate, such as plastic or TCT, while maintaining an optimal cell growth area and without the need for a retainer device, such as an “O-ring” to mechanically hold the material to the underlying plastic support. Thus, for example, a LBG only coating on a TCT surface delaminates from the underlying TCT surface. Mechanical clamping of the LBG coating to the underlying substrate using, for example, an “O-ring” can prevent delamination. However, it is preferable to provide a coated substrate which is free of any unnecessary parts or processing step. Microscope images of delamination in LBG alone compared to no delamination in a blended material are shown in FIGS. 4A and 4B, respectively.

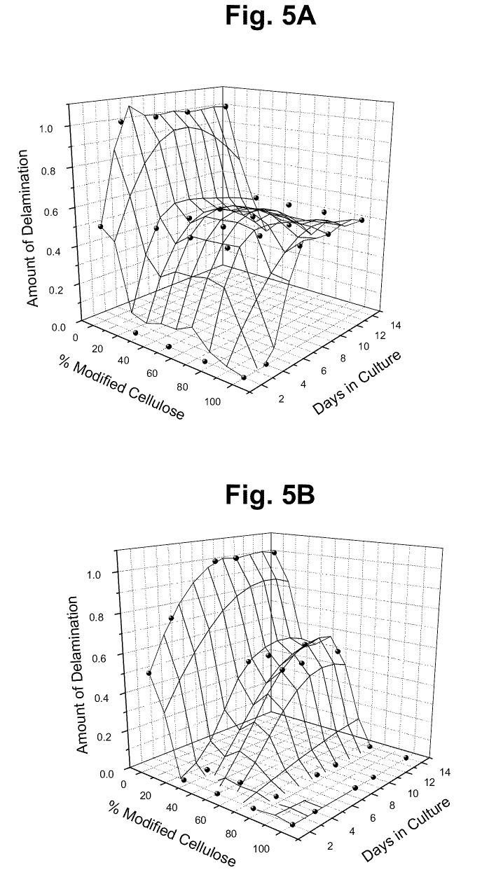

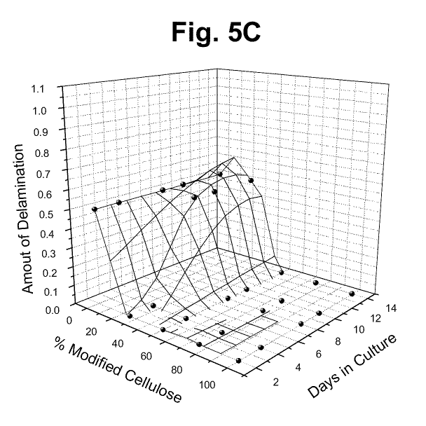

Blends were prepared and assessed for amount or extent of delamination. A rank of 1 was given to a material that completely delaminated, a rank of 0.5 was given to a blend that partially delaminated, and a rank of 0 was given to a blend that did not delaminate. Rank determinations were made by viewing the material under an optical microscope with a 5× magnification objective using brightfield. Delamination was monitored as a function of % (y volume) added hydrophobically modified cellulose and time in culture for 3 blends: LBG:N100; LBG:N430; and LBG:PS67; against base control materials LBG, N100, N430 and PS67. Delamination response curves of LBG:N100, LBG:N430 and LBG:PS67 blends are shown in FIGS. 5A to 5C, respectively. FIGS. 5A to 5C show a sample data set for each condition that was monitored. The dots in these figures represent the actual data, while the gridlines are included to aid comprehension.

Certain commercially available substrates, such as Matrigel® and Collagen I, for hepatocyte culture may provide for cell attachment but not for optimal cell attachment, such as the total number of cells attached. For cell harvesting and assay considerations, it is desirable to have large numbers of cells attached to the substrate.

Modified cellulose (such as hydroxyethyl cellulose, hydroxypropyl cellulose, methylcellulose or derivatives thereof) has been used (see ref 5) as an associative thickener, emulsifier, and rheology modifier in a variety of industries including food, pharmaceutical, personal care, and paint. In addition, Methocel® (which is a family of cellulose ethers that include water-soluble methylcellulose and hydroxypropyl methylcellulose polymers commercially available from Dow Chemical) films or like hydroxymethyl, hydroxyethyl, or hydroxypropyl cellulose materials, are cell adhesion resistant materials and are used for anchorage independent assays (see refs. 6-7).

HEC functional group modification. Additionally or alternatively, polysaccharides of the disclosure can be further functionally modified with, for example, urethanes, maleimides, esters, functionalized with biological macromolecules, or like groups for increasing or decreasing specific intra- or interchain interactions of hydrophobically modified groups.

The hydroxyl groups of cellulose can be partially or fully reacted with various reagents to afford derivatives with useful cell culture coating properties. Cellulose esters and cellulose ethers, for example, are readily available commercial materials. Lower molecular weight modified cellulose esters are known, such as cellulose acetate and cellulose triacetate, which are known film- and fiber-forming materials having various industrial uses. Ether modified cellulose derivatives can be selected as the hydrophobe modified polysaccharide or used as starting material for further hydrophobic derivatization and can include, for example, alkycellulose, such as incompletely alkylated methyl or ethyl cellulose. Other suitable cellulose starting materials can include, for example, hydroxypropyl cellulose, carboxymethyl cellulose, hydroxypropyl methyl cellulose, hydroxyethyl methyl cellulose, and like materials, or combinations thereof.

HMHEC alkyl ethers, hydroxy alkyl ethers, esters, and like hydrophobically linked HECs are commercially available or can be readily prepared using various reactants, see for example, the working examples and above mentioned PolySurf® 67 chemical structure and refs. 8. If desired one can append biologic molecules of interest such as peptides, growth factors (such as HGF), antioxidants, glycans, or like molecules to the hydrophobe modified polysaccharide(s) to further modify substrate coating surface properties, such as substrate adhesion, cellular attachment, cell function, cell proliferation, coating release, and cell release properties. The polysaccharide polymer can be further modified using, for example, standard carbohydrate and polymer functionalization techniques to attach a variety of (macro)molecules. For example, one can incorporate a maleimide functional group using a Mitsunobu reaction (PPh₃, diethyl azodicarboxylate as catalyst) to easily and selectively attach a thiol-containing molecule, such as cysteine modified polypeptide, to the polymer. Alternatively, any thiol containing compound can be used to add another functional group of interest if a spacer is desired or necessary for proper ligand display.

The abundance of hydroxy groups in the HMHEC allows for polymer modification using ester formation using, for example, coupling reagent combination such as EDC/NHS (N-(3-Dimethylaminopropyl)-N′-ethylcarbodiimide and N-hydroxysulfosuccinimide), or like carbodiimide coupling reagents. A preactived carboxylic acid such as a succinimide ester or p-nitrophenol carbonate ester can also be used in the absence of the coupling reagents.

Any of the aforementioned or like chemistries (ref. 4) can also be used to further modify the polymer with hydrophobe groups. This modification can increase the hydrophobic interaction(s) between the polysaccharide chains and permit tuning of useful cell culture time and coating release from the substrate. Other self-associating groups, such as perfluoroalkyl or silicone, can be incorporated into the coating formulation to provide additional coating cohesion, but not cell adhesion, particularly if there is an interest in probing specific ligand-receptor interactions.

Additionally or alternatively, polysaccharides of the disclosure and like materials can be functionally modified using, for example, maleimide chemistry, esterification, functionalization of biological macromolecules, or like methods for increasing specific intra- or interchain interactions of hydrophobically modified groups. If desired one can append biologically relevant molecules such as peptides, growth factors (such as HGF), antioxidants, glycans, or like entities to the cellulose polymers to modify, for example, cellular attachment, function, and proliferation.

Alternative polymers Other water soluble polymers including, for example, poly(vinyl alcohol), poly(acrylamide), poly(acrylic acid), and linear or branched polysaccharides including, for example, dextran, agarose, chitosan, amylose, amylopectin, and like polymers, can be selected as additives or can be chemically modified with hydrophobic or like associating groups, including self-complexing and complementary binding pairs, to produce tunable, for example, viscoelastic coatings having hydrogel-like properties that are suitable for cell culture and assay. Further fine tuning of the substrate coating properties with respect to, for example, the cell type, assay, and release, can be accomplished by, for example, combining one or more of the examples mentioned above or disclosed herein.

The disclosure provides in embodiments compositions for cell culture surface modification including a combination or blend of a hydrophobically or hydrophobe modified hydroxyethyl cellulose (HMHEC), such as Natrosol® and PolySurf®, and another cell culture substrate media, such as a natural or synthetic gum, or a water swellable polymer. In embodiments, the combination of HMHEC and another cell culture substrate surface media (“combined surface media”) can enhance, for example, the overall performance of the substrate, the cell culture, or both, by providing superior adhesion of the combined surface media to the underlying support, such as a plastic mass or monolith, or plastic coated monolith, as found in, for example, plastic tissue culture labware, or like articles. In embodiments, the combined surface media when applied to a cell culture support or surface can produce desirable surface roughness characteristics, for example, a surface roughness that facilitates or promotes increased cell attachment, enhanced cell morphology, and provides substrate clarity. In embodiments, the disclosure also provides methods for culturing anchorage-dependent cells, including for example hepatocytes, in vitro. The disclosed combined culture media can be particularly useful for hepatocyte or other mammalian (e.g., primary, cancer, etc.) cells that require a lower complex bulk modulus compared to, for example, polystyrene. In embodiments, the disclosed surfaces provide for greater cell-cell attachment and communication and achieve approximate in vivo like function. Matrigel®'s hulk complex modulus is approximately 10-100 Pa at 40° C. and at shear rates of 0.1 to 100 rad/s. In this context “lower” refers to modulus measurements that are about 10-100 Pa for complex modulus as defined above. Examples of cell culture media materials having a lower modulus are illustrated in Table 1. Locust bean gum in contrast has a high bulk complex modulus of about 335-540 Pa.

TABLE 1
Cell culture coating materials having low complex modulus.

| | Complex Modulus |
|---|---|
| Cell Culture | G* [Pa] (0.1-10 rad/s) |
| Coating | @ 37° C. |
| Matrigel ® | 7-16 |
| Collagen gel | 13-25 |
| Cassia gum | 5-13 |
| Agarose 0.5% | 70.5-100 |

In embodiments the disclosure provides a cell culture substrate or article having superior cell maintenance and cell function properties, such as albumin production, compared to other commercially available products, for example, Matrigel® or completely hydrophilic materials, such as Collagen I.

In embodiments the disclosure provides a cell culture substrate coating having surface attachment points that may be particularly conducive for anchorage dependent cells. Although not bound by theory, it is believed that for example, ASGP receptors on hepatocyctes interact with the galactose side chain of the substrate coating as suggested by fluorescence microscopy (photo not shown). The disclosed blends can provide substrate coatings that are substantially optically clear and can provide cell culture articles that are easy to examine or analyze, such as visually or optically.

The disclosed blends provide substrate coatings that are non-animal derived and the resulting coated products with, for example, less or no lot-to-lot variability. The disclosed blends provide substrate coatings that are more economical, such as for example about 10 to about 50% less costly, compared to substrate coatings of only LBG. The disclosed blends provide substrate coatings that can easily incorporate attachment points to covalently link, for example, macromolecules to enhance attachment, enhance function, or reduce oxidative damage arising from, for example, CYP450 activity. The disclosed blends provide substrate coatings having a modulus that can be easily tuned by chemical techniques, for example, cross-linking such as by UV or thermally curable modifications or esterification. The disclosed blends provide substrate coatings that are non-toxic and biocompatible. The disclosed blends provide substrate coatings that are highly processible, such as easy to deposit onto a variety of surfaces, and provide enhanced adhesion of the coating and of cells to plastic substrates.

The disclosure relates to the use of a hydrophobically modified cellulose polymer in combination with another component, for example as an additive, to surface-coat cell culture media to leverage the hydrophobically modified cellulose polymer's: 1) compatibility with water soluble, swellable, hydrogel polymers that can result in enhanced adhesion to, for example, plastic or like substrates, such as injection molded articles, and 2) immiscibility with other water swellable polymers to impart texture to the cell culture coating. Surprisingly and unexpectedly, the observed increased surface texture or topography of the blend-coated substrates provided increased cell adhesion or cell attachment to the coated substrates despite using a known cell attachment resisting material.

Examples of hydrogel polymer materials can include, for example, gums such as locust bean gum, cassia gum, guar gum, carboxymethylguar gum, fenugreek gum, mesquite gum, and like gums, or combinations thereof.

In embodiments, the disclosure provides methods for cell culture of, for example, anchorage-dependent mammalian cells. The disclosure can also be adapted to, for example, cancer cell lines and anchorage independent cell assays by selection of specific polymer blends, for example a 50:50 or 1:1 (vol:vol) of a locust bean gum:Natrosol® 430 combination, or like combinations. The resulting blended material can be used as-is to produce thin film surfaces, for example, about 0.003 to about 0.005 cm, to culture cells or as a gel to encapsulate cells or cell clusters. In embodiments, the disclosure provides methods for cell culture of, for example, encapsulated cells or cell clusters, see for example, U.S. Patent Publication No. 2007/0148767.

### EXAMPLES

The following examples serve to more fully describe the manner of using the disclosure, as well as to further illustrate and demonstrate specific examples of best modes contemplated for carrying out various aspects of the disclosure. These examples do not limit the scope of the disclosure, but rather are presented for illustrative purposes. Compositions or mixtures of materials that mention percents or parts refer to weight percents (wt %) or parts-by-weight unless specifically indicated otherwise, such as mol %.

### Example 1

Blend Preparation, Coating and Cell Culture Procedures. For sequentially formed blends with locust bean gum, a 0.2 wt % Natrosol® 430 in water solution was plated in a 24-well plate format on a tissue culture treated polystyrene substrate followed by a 1 wt % aqueous solution of locust bean gum (Fluka #62631). Both the Natrosol® and the locust bean gum were separately dissolved in deionized water. Stock 0.2 wt % Natrosol® solutions were stirred about 16 hrs at room temperature to allow complete dissolution. Stock 1 wt % locust bean gum aqueous solutions were heated to between 70 and 90° C. to allow dissolution of the gum material prior to coating the same day. To make blended materials such as 50:50 (vol:vol) Natrosol®:gum, 500 microL of the 0.2 wt % Natrosol® in water was applied to the microplate followed by 500 microL of 1 wt % locust bean gum in water to make a total of 1 mL solution prior to coating. Coatings were applied using a pipette onto a tissue culture treated surface. After sequential application of the coatings, the coatings were mixed in the well with a plate shaker for 30 sec to ensure complete well coverage and a homogeneous mixture. Alternatively, the separate coating solutions could be mixed together before application to the well, followed by use of a plate shaker as described above. The coatings were dried at 60° C. overnight, UV sterilized (365 nm) for 1 hour, and subsequently used for cell culture. HepG2/C3A cells (ATCC # CRL-10741) were cultured in Eagle's Minimum Essential Medium (“EMEM”, ATCC #30-2003) supplemented with 10% Fetal Bovine Serum (Invitrogen #16000-077) and 1% penicillin-streptomycin (Invitrogen #15140-155) at 20 k cells/well. Cells were incubated at 37° C., in 5% CO₂ and 95% relative humidity. The supplemented EMEM liquid medium was replaced every other day. Albumin levels were determined using Micro-Albumin Quantitative Test (Biomeda # EU-1057).

For E-Cadherin staining, cells were washed and fixed using paraformaldehyde (4% in phosphate buffer saline (PBS)) for 10 mins, followed by permeabilization for 10 mins using 0.1% Triton X-100. Cells were incubated with 1% bovine serum albumin (BSA) in PBS for 30 min, then with 1:200 dilution of mouse anti-E-Cadherin antibody in PBS and 1% BSA. After about 16 hours incubation under cell culture conditions, the cells were washed twice with PBS, followed by incubation at 1:100 dilution with Cy3-Goat anti-mouse IgG conjugate in PBS with 1% BSA for 30 min, and finally rinsed twice with PBS. The resultant cell samples were imaged with a fluorescence or confocal microscope.

To prepare blends containing (LMA-HEMA) copolymer (a random copolymer of lauryl methacrylate (LMA) monomer and 2-hydroxyethyl methacrylate (HEMA) monomer), Solutions of Natrosol® or Methocel® were prepared as 0.2 and 0.1 wt % solutions, respectively, in deionized water. A 10% (LMA-HEMA) copolymer solution in ethanol was prepared and then further diluted with ethanol to make 1 wt % solutions. The blends were prepared by separately adding the aqueous solutions of Natrosol® or Methocel® to the ethanolic solution of the (LMA-HEMA) copolymer to achieve about 10% or about 20% cellulose content relative to the (LMA-HEMA) copolymer. The resulting clear solution was then added to 96-well plates (100 microL per well) and dried in a 60° C. oven. The coated substrates can be stored at room temperature without special precautions or treatments.

The (LMA-HEMA) copolymers were prepared by free radical polymerization in ethanol using AIBN as the initiator. For a related preparative procedure see for example, I. Erol, in J. App Polym Sci, Vol. 100 1864-1874 (2006). The reaction solution was about 10 wt % in total monomer and heated to about 60° C. for about 16 hrs. The copolymers were purified by precipitation. Lauryl methacrylate monomer is commercially available from Rohm and Haas (such as ROCRYL™ 320 Lauryl Methacrylate (LMA) Alkyl ester monomer, formula weight 260, The 2-hydroxyethylmethacrylate (HEMA) monomer is commercially available, for example from Sigma-Aldrich (St. Louis, Mo.). Mole ratios of exemplary (LaurylMA-HEMA) copolymers are contained in Table 2.

TABLE 2
Mole ratios of exemplary (LMA-HEMA) copolymers.

| LMA | HEMA |
|---|---|
| monomer | monomer |
| 25 | 75 |
| 10 | 90 |

TABLE 3
Molecular weight properties of (LMA-HEMA) copolymers.

| (LMA-HEMA) | Weight Average | Number Average | |
|---|---|---|---|
| copolymer | Molecular Weight | Molecular Weight | Polydispersity |
| Mol % | (M_{w}) | (Mₙ) | (M_{w}/Mₙ) |
| 1% lauryl¹ | 3,200 | 1,700 | 1.93 |
| 5% lauryl | 15,600 | 8,000 | 1.95 |
| 10% lauryl | 41,000 | 16,800 | 2.45 |
| 25% lauryl | 73,000 | 19,800 | 3.68 |
| 50% lauryl | 110,000 | 39,400 | 2.80 |
| 75% lauryl | 91,400 | 26,800 | 3.41 |
| 100% lauryl | 90,500 | 21,600 | 4.20 |

¹This material was difficult to dissolve in THF.

### Example 2

Hydrophobic modification of hydroxyethylcellulose (HMHEC)

Materials: hydroxyethylcellulose (“HEC”; Dow QP30), acid chloride functionalized alkyl group (or other hydrophobic groups), NMP, isopropyl alcohol (IPA) and acetone.

Procedure: The hydroxyethylcellulose was pre-dried in a desiccator for about 16 hours. The hydroxyethylcellulose was dissolved in NMP (e.g., about 100 mg HEC/5 mL NMP). Dissolution can typically be facilitated with gentle warming, stirring, or both. Next, the acid chloride is diluted with NMP to obtain a workable solution or suspension. The acid chloride solution was added dropwise with stirring to the hydroxyethylcellulose and NMP solution and stirred at room temperature for about 2 hours or more. The reaction mixture was poured into acetone and stirred in a closed container to exclude moisture for about 30 min to precipitate a product. At higher hydrophobic modifications the acetone solvent can be mixed with or replaced by, for example, methanol. The crude precipitate was filtered and washed with acetone (or like wash solvent) then dried at 60° C. in an oven or a heated or ambient vacuum desiccator. In instances where greater product purity is desired, the product can be dissolved in, for example, NMP or other suitable dissolving non-aqueous solvent or solvent mixtures, to reprecipitate. The isolated product can be stored in a sealed container at room temperature.

Example: HEC (300 mg) in NMP (15 mL); Lauryl acid chloride stock solution (0.5 mL in 9.5 mL NMP); About 0.1 mL of lauryl acid chloride stock solution was added to the HEC. The isolated product, lauryl-HEC, was off-white powder, and was obtained in an isolated yields of about 90%.

### Example 3

Copolymerization of alkylacrylate and HEM monomers About 100 mmol of an alkyl methacrylate monomer, such as lauryl methacrylate, and about 100 mmol of a hydroxyalkyl methacrylate monomer, such as HEMA, were measured into a 20 mL scintillation weighing vial. The monomer mixture was added to a 500 mL 2-neck flask equipped with a gas outlet tube and fitted with a condenser linked to a nitrogen source and addition funnel for adding reagents, and a magnetic stirrer. 125 mL of absolute ethanol was used to transfer residual monomer remaining in the weighing vial to the reaction flask. The monomer solution was stirred and the flask purged with nitrogen by first evacuating under vacuum and then filling the flask with nitrogen and the sequence was repeated. Free radical initiator AIBN (130 mg) was added in a single batch. The flask was heated to and maintained at 60° C. with stirring for 16 hours under N₂. The reaction mixture was cooled to room temperature and 125 mL of isopropyl alcohol was optionally added. Next, the reaction mixture was slowly added to a vigorously stirred flask containing 2 L of precipitating solvent, such as hexanes. The resulting precipitate was filtered, briefly air dried, then dried in a 60° C. oven for 16 hours to remove substantially all residual solvent. The polymer product was ground-up in a coffee grinder for ease of handling, and stored at room temperature until used further.

### Example 4

Hydrogel coatings including ARC to enhance adhesion A 0.2% by weight hydrophobically modified cellulose polymer, such as Polysurf 67® or Natrosol® 430, was dissolved in 1 mL of deionized water. About 500 microliters of this solution was then coated on a 24-well plate, or any suitable cell culture vessel including, for example, flasks, Petri dishes, well pates, slides, and like vessel surfaces, using standard coating methods, such as drop casting, dip coating, spin coating, and like methods, or a combination thereof. A 1% by weight of a hydrogel former, such as locust bean gum, was dissolved in 1 mL of deionized water by heating to about 70-90 ° C. About 500 microliters of this solution was then subsequently coated on the abovementioned hydrophobically modified cellulose coating to make a 50:50 blended and overcoated thin film having an overall thickness of about 0.003 to about 0.005 cm after being dried at 60° C. for about 16 hrs. Alternatively, the two solutions can be mixed together and coated or cast onto the cell culture substrate to form a true blended coating. The blended materials appear to be more optically clear, by visual inspection and by microscopic visible light transmission compared to the films cast from the separate components.

In some instances, hydrophobe modified cellulose polymers of higher molecular weight can further enhance adhesive properties of the coating to the substrate. The hydrophobic groups of Natrosol® or Polysurf® type polymers can promote better adhesion of the coating material to the substrate surface, such as polystyrene, or like plastic surfaces including for example polyolefin, polypropylene, like polymers, and copolymers thereof, and prevent delamination of the hydrogel coating from the surface. Cell cultures having the coated surfaces were evaluated in Example 5.

Additionally or alternatively, the hydrogel blended material can be used to create a sandwich culture by coating, such as by pouring or like coating methods, a second layer of hydrogel blend on top of the cells seeded on the first hydrogel blend coating if this configuration is beneficial for a particular cell type or assay.

### Example 5

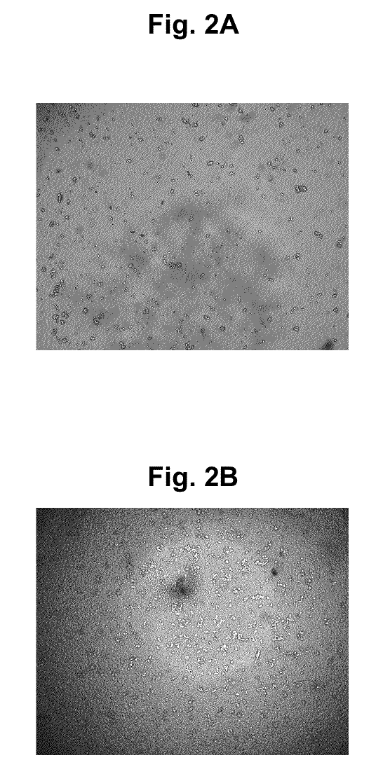

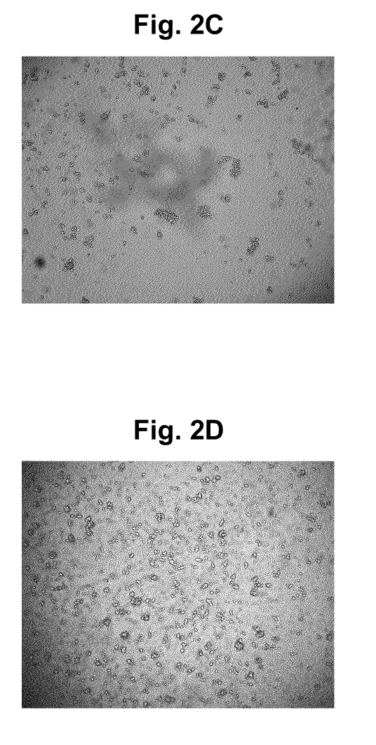

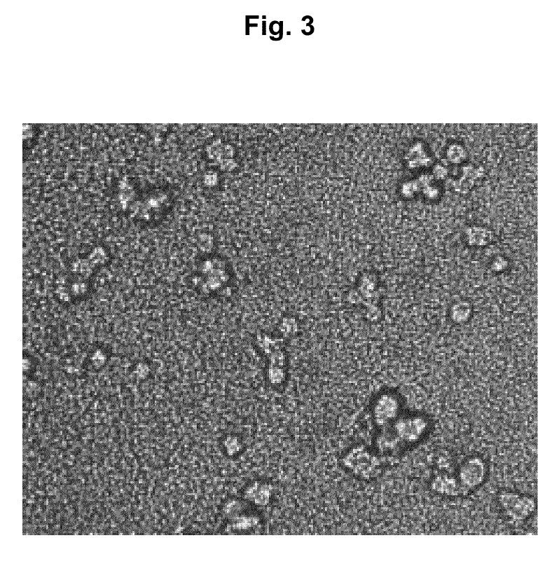

Topography Natrosol® and other water soluble cellulose polymers (such as Methocel® and Ethocel®) can also be used as an immiscible phase to impart texture to the cell culture material of interest. For example, random copolymers of monomers, such as lauryl methacrylate and 2-hydroxyethyl methacrylate containing about 10 mol percent of lauryl methacrylate, produce relatively featureless coatings when cast from ethanol (1 wt % solution). However, adding 10 to 20 wt % water soluble cellulose polymer relative to the lauryl-HEMA copolymer as an aqueous solution gave a surface that was transparent but was rougher, see FIG. 2. The textured surfaces (e.g., FIG. 3) allowed more cells to attach relative to either the lauryl-HEMA copolymer or Methocel® but retained the same more “rounded” cellular morphology. This cell shape is indicative of weaker attachment to the substrate and allows for a more 3D (i.e., not flattened and spread-out) cell morphology, which is highly desirable for some cell types for enhanced function.

### Example 6

Cell Culture Cells (HepG2/C3A) cultured on HMHEC, such as Natrosol® 430/LBG blends (locust bean gum available from Fluka or Sigma) form multicellular aggregates that show E-cadherin staining similar to those found in Matrigel.® Images of stained hepatocyte cells cultured on blend coated substrates of the disclosure and a commercially coated substrate were obtained and compared. A hepatocyte cluster was cultured on a blend coated substrate of a 50:50=vol:vol blend of Natrosol® 430:locust bean gum and E-Cadherin stained. A comparative hepatocyte cluster was cultured on Matrigel® and E-Cadherin stained. Both images (photos not included) showed characteristic gap junctions which are indicative of good hepatocyte cell function.

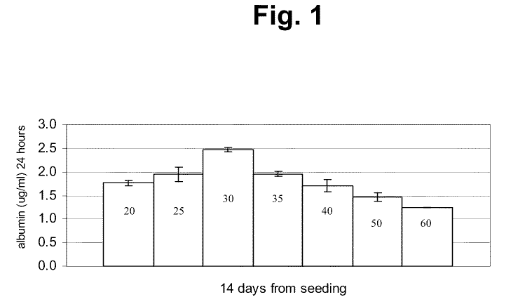

Referring to the Figures, a number of the disclosed blends show improved function, such as improved albumin production, as shown in FIG. 1. FIG. 1 shows actual and comparative results of albumin production of hepatocyte cells cultured on substrates having different surface coat compositions. Albumin production of HepG2/C3A cells at day 14 cultured on locust bean gum (LBG)(20), blends of LBG with Natrosol® 430 (N) (LBG/N: 75:25 (vol:vol) (25); 50:50 (vol:vol) (30); 25:75 (vol:vol) (35)), Natrosol® 430 (N430) alone (40), and on tissue culture treated (50) (TCT) (plasma treated polystyrene from Corning Inc.) and Matrigel® (60) standards.

### Cell Adhesion Image Analysis

Image analysis was performed to estimate the increase in hepatocyte cell attachment to coated substrates of blended materials (e.g., 10 wt % and 25 wt % LaurylMA-HEMA copolymer combined with Methocel® K4M) compared to coated substrates of 10 wt % and 25 wt % LaurylMA-HEMA copolymer alone. Similar results were also observed for blends formulated with Methocel® F240 in place of Methocel® K4M. Methocel® K4M alone was not compared since it is known to be non-attaching. The number of cells in each image was estimated by surface area coverage of cells versus unoccupied areas after 1 day of culture (i.e., cell attachment and not cell proliferation). The images were obtained with a light microscope (brightfield) using a 5× objective. The analyzed images were cropped to reduce lighting artifacts, such as observed at edges of the images. FIGS. 2A to 2D shows magnified (5× brightfield micrographs) images of a LMA-HEMA copolymer control coatings compared to exemplary coatings consisting of blends of LMA-HEMA copolymer and Methocel®. The micrograph results demonstrated enhanced cell attachment characteristics. FIG. 2A shows surface area coverage of cells for a control coating made from a 10 wt % solution of a 25:75 mol % LMA-HEMA copolymer. The surface area coverage of cells on the 10 wt % control coating was about 6.8%. FIG. 2B shows surface area coverage of cells on a coating made from a blend of a solution of a 10 wt % LMA-HEMA copolymer and 20 wt % Methocel® K4M which was about 16% which represents an increase in cell surface area coverage of about 235%, that is, about 235% more cells adhered to the blend surface compared to the LMA-HEMA copolymer alone. FIG. 2C shows a control coating made from a 25 wt % LMA-HEMA copolymer solution. Comparable cell surface area coverage results were observed (FIG. 2D) for a blend coating made from a solution of 25 wt % LMA-HEMA copolymer and 20 wt % Methocel® K4M as was observed in FIG. 2B.

FIG. 3 shows a magnified (20×) brightfield image of the abovementioned FIG. 2D that flirter shows cell attachment as clustered structures, and coated substrate roughness or topography as the mottled background. Although not bound by theory the uniformly mottled appearance of the coated substrate is believed to be topography due to phase separation of the blended materials.

FIG. 4A shows an example of a magnified image of locust bean gum coating delamination on a TCT substrate. The non-delaminating LBG on TCT substrate occupies the left-hand and lower two-thirds and the delaminating LBG coating occupies the right-hand and upper one-third of the image. Separation of the LBG coating occurs leaving uncoated TCT surface, for example, when the LBG coating is exposed to water. FIG. 4B shows an example of a more robust, non-delaminating, coating of a 1:1 weight ratio blend of locust bean gum and Natrasol® 430, including viable cells, that occupy the right-hand and upper one-third of the image. The left-hand and lower two-thirds of image FIG. 4B represents the edge of the cell culture well.

FIGS. 5A-5C show delamination response curves for several LBG:hydrophobe modified polysaccharide blends (FIG. 5A: LBG: Natrosol® 100 from 75:25 (vol:vol) to 25:75 (vol:vol); FIG. 5B: LBG: Natrosol® 430 from 75:25 (vol:vol) to 25:75 (vol:vol); and FIG. 5C: LBG: PolySurf® from 75:25 (vol:vol) to 25:75 (vol:vol) as a function of time in cell culture and % added hydrophobically modified cellulose.

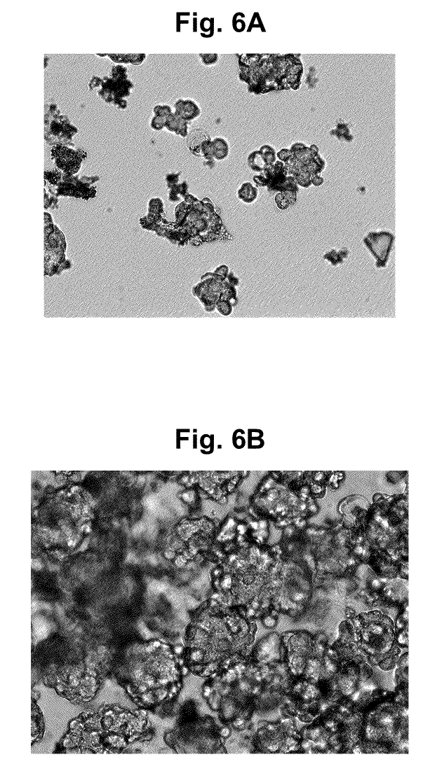

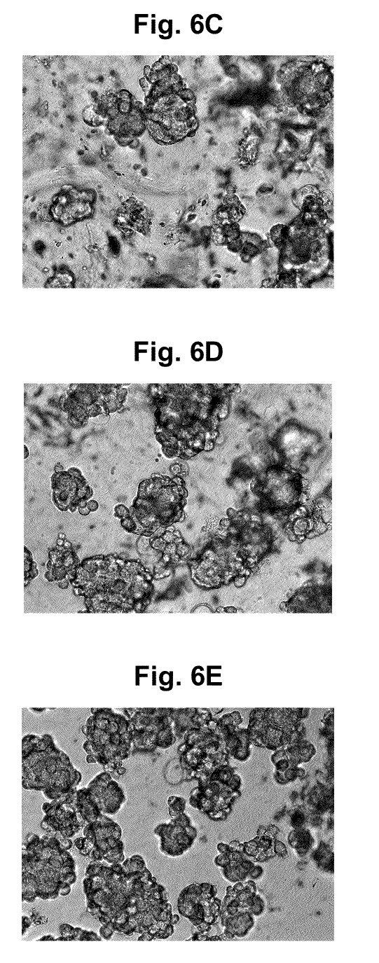

FIG. 6 show examples of magnified (20× micrographs) images of controls and several exemplary coatings consisting of admixtures of locust bean gum and Natrasol® 100. FIG. 6A is a 0.2 wt % coated solution of Natrosol® 100 alone showing viable cell clusters and an optically clear or transparent background. FIG. 6B is a 1.0 wt % coated solution of LBG alone showing viable cell culture and a slightly opaque or optically occluded background. FIG. 6C is a coated solution of a blend of 75:25 (vol:vol) LBG: Natrosol® 100 showing cell culture clusters and an opaque or optically occluded background. FIG. 6D is a coated solution of a blend of 50:50 (vol:vol) LBG: Natrosol® 100 also showing cell culture clusters and a more clear or optically transparent background compared to the 75:25 (vol:vol) LBG: Natrosol® 100 blend. FIG. 6E is a coated solution of a blend of 25:75 (vol:vol) LBG: Natrosol® 100 showing cell culture clusters and an even clearer or more optically transparent background compared to the 50:50 (vol:vol) LBG: Natrosol® 100 blend.

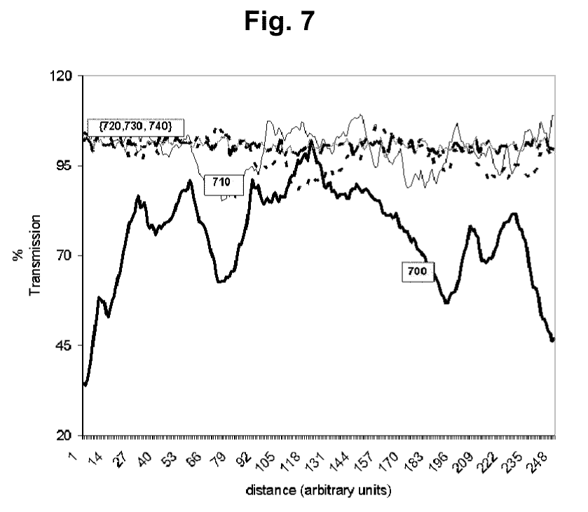

FIG. 7 shows a chart of % transmission vs distance as a measure of optical clarity as determined by a line scan in a cell-free area in the samples examined in LBG, N100, and the LBG:N100 blended materials of FIG. 6. Optical clarity was determined by (20×) optical micrographs of, for example, HepG2/C3A cells at day 3 of culture on LBG (700) control, N100 (740) control, and their blends: 25/75 (vol:vol) LBG/N100 (720), 50/50 (vol:vol) LBG/N100 (730), and 75/25 (vol:vol) LBG/N100 (710). Gray levels were assessed in the micrographs in a region without cells to determine the % transmission of the cell culture substrate. The LBG control had the lowest % transmission. The 75/25 (vol:vol) LBG/N100 (710) had the next lowest but improved % transmission compared to the LBG (700) control. The 25/75 (vol:vol) LBG/N100 (720), 50/50 (vol:vol) LBG/N100 (730), and N100 (740) control had substantially 100 percent transmission.

The disclosure has been described with reference to various specific embodiments and techniques. However, it should be understood that many variations and modifications are possible while remaining within the spirit and scope of the disclosure.

### REFERENCES

1. See generally: http://www.cyprotex.com/products/ADME%20Guide%20.pdf in Cyprotex publication library for general ADMETox information; and Vanhaecke, T., et al., “Hepatocyte Cultures in Drug Metabolism and Toxicological Research and Testing,” Cytochrome P450 Protocols in Methods in Molecular Biology Series, and Phillips, I., et al., Eds. Methods in Molecular Biology Series 320, Humana Press Inc., Totowa, N.J., 2006, pp. 209-227.
2. LeCluyse, E. L. European J. Pharm. Sci., 2001, 13, 343-368.
3. Moghe, Prabbas V., et al., “Culture matrix configuration and composition in the maintenance of hepatocyte polarity and function,” Biomaterials, 1996, 17, 373-385.
4. Commonly owned and assigned copending patent application, U.S. Provisional Application Ser. No. 60/906,168, filed Mar. 9, 2007, entitled “Coatings for Cell Culture Surfaces” (SP07-055P).
5. Hercules (www.herc.com/aqualon/aq_markets.html)
6. http://www.lbl.gov/lifesciences/BissellLab/labprotocols/anchorage.htm (Lawrence Berkeley Laboratories, Life Sciences Division)
7. Zhang, L.-M., Carbohydr. Polym., 2001, 45, 1-10.
8. Kawakami, et. al., “Salt Tolerance of an Aqueous Solution of a Novel Amphiphilic Polysaccharide Derivative,” Langmuir, 2006, Vol. 22, No. 7, 3337; Ihara, et. al., “Solution Properties of a Novel Polysaccharide Derivative,” Chem. Lett, 2004, Vol. 33, No. 9, 1094 (mentions a hydrophobically (stearyl alkyl group) modified hydroxyethylcellulose, R-HEC, a glycidal ether and stearyl alcohol adduct); Akiyama, et al., J. Colloid Interface Sci., 2005, 282, 448.

What is claimed is:

1. A cell culture composition comprising:
a hydrophobe modified polysaccharide comprising a polysaccharide having a plurality of hydrophobes, the hydrophobe having from 6 to about 22 carbon atoms, the hydrophobes comprise from about 1 to about 20 mole % and the polysaccharide comprises from about 80 to about 99 mole %; and
a water soluble, swellable hydrogel that is miscible with the hydrophobe modified polysaccharide.

2. The composition of claim 1 wherein the hydrophobe comprises a C₁₂₋₂₀ alkyl and the polysaccharide comprises a cellulose.

3. The composition of claim 1 wherein the hydrophobe is selected from the group consisting of:
—O—R;
—S—R;
—O—C(═O)R;
—S—C(═O)R;
—S—C(═S)R;
—C(═O)R;
—O—CH₂—CH(OH)—CH₂—O—R;
—O—CH₂—CH(OH)—CH₂—O—C(═O)R; or
—O—CH₂—CH(OH)—CH₂—C(═O)R,
and combinations thereof, where R is a C₁₆₋₂₀ alkyl having a linear alkyl chain, a branched alkyl chain, or a mixture thereof, and the polysaccharide comprises a cellulose, an hydroxyalkyl cellulose, an oligomeric (hydroxyalkyl) cellulose, or mixtures thereof, the hydroxyalkyl celluloses having from 0 to about 3 hydroxyalkyl groups for each saccharide of the polysaccharide, the hydroxyalkyl group having from 2 to 6 carbon atoms.

4. The composition of claim 1 wherein the polysaccharide comprises a hydroxyethyl modified cellulose having from 0 to 3 hydroxyethyl groups for each glucose, the hydrophobe comprises a —O—C(═O)R, where R is C₁₆₋₂₀ alkyl, and having a total hydroxyethyl to hydrophobe mole ratio of from about 6:1 to about 1:1.

5. The composition of claim 1 wherein the polysaccharide comprises a hydroxyethyl modified cellulose having from 1 to 3 hydroxyethyl groups for each glucose, the hydrophobe comprises a —O—C(═O)R, where R is C₁₈ alkyl, and having from about 2 to about 15 mole % hydrophobe and from about 85 to about 98 mole % polysaccharide.

6. The composition of claim 1 wherein the cell culture composition comprises:
from about 10 to about 90 wt % a hydrophobe modified cellulose; and
from about 10 to about 90 wt % water soluble, swellable hydrogel.

7. The composition of claim 1 wherein the water soluble, swellable hydrogel comprises at least one galactomannan gum selected from locust bean gum, carob bean bum, carob seed gum, carob gum, guar gum, cassia gum, tara gum, mesquite gum, fenugreek gum, or a combination thereof.

8. The composition of claim 1 wherein the hydrophobe modified cellulose promotes adhesion of the hydrogel to the substrate.

9. The composition of claim 1 wherein films of the composition are optically clear.

10. A cell culture composition comprising:
a hydrophobe modified polysaccharide comprising a polysaccharide having a plurality of hydrophobes, the hydrophobe having from 6 to about 22 carbon atoms, the hydrophobes comprise from about 1 to about 20 mole % and the polysaccharide comprises from about 80 to about 99 mole %; and
a water swellable polymer that is immiscible with the hydrophobe modified polysaccharide.

11. The composition of claim 10 wherein the hydrophobe comprises a C₁₂₋₂₀ alkyl and the polysaccharide comprises a cellulose.

12. The composition of claim 10 wherein the hydrophobe is selected from the group consisting of:
—O—R;
—S—R;
—O—C(═O)R;
—S—C(═O)R;
—S—C(═S)R;
—C(═O)R;
—O—CH₂—CH(OH)—CH₂—O—R;
—O—CH₂—CH(OH)—CH₂—O—C(═O)R; or
—O—CH₂—CH(OH)—CH₂—C(═O)R,
and combinations thereof, where R is a C₁₆₋₂₀ alkyl having a linear alkyl chain, a branched alkyl chain, or a mixture thereof, and the polysaccharide comprises a cellulose, an hydroxyalkyl cellulose, an oligomeric (hydroxyalkyl) cellulose, or mixtures thereof, the hydroxyalkyl celluloses having from 0 to about 3 hydroxyalkyl groups for each saccharide of the polysaccharide, the hydroxyalkyl group having from 2 to 6 carbon atoms.

13. The composition of claim 10 wherein the polysaccharide comprises a hydroxyethyl modified cellulose having from 0 to 3 hydroxyethyl groups for each glucose, the hydrophobe comprises a —O—C(═O)R, where R is selected from a C₁₆₋₂₀ alkyl, and having a total hydroxyethyl to hydrophobe mole ratio of from about 6:1 to about 1:1.

14. The article of claim 10 wherein the cell culture composition comprises: from about 5 to about 25 wt % a hydrophobically modified cellulose; and from about 75 to about 95 wt % a water swellable polymer.

15. The composition of claim 10 wherein the water swellable polymer comprises at least one of copolymer comprised of a hydroxyalkyl methacrylate monomer and an alkylmethacrylate monomer.

16. The composition of claim 10 wherein the water swellable polymer comprises a copoly(laurylmethacrylate-2-hydroxyethylmethacrylate).

17. The composition of claim 10 wherein the hydrophobe modified polysaccharide in combination with the water swellable polymer promotes increased cell adhesion to a substrate coated with the combination, and the water swellable polymer imparts surface topography to the coated substrate.

18. A cell culture article comprising:
the composition of claim 1 coated on a substrate.

19. The article of claim 18 wherein the coating further comprises a biologically active compound comprising at least one of an amino acid, a peptide, a polypeptide, a protein, a carbohydrate, a lipid, a polysaccharide, a nucleic acid, a nucleotide, a polynucleotide, a glycoprotein, a lipoprotein, a glycolipid, a glycosaminoglycan, a proteoglycan, a growth factor, a differentiation factor, a hormone, a neurotransmitter, a pheromone, a chalcone, a prostaglandin, an immunoglobin, a monokine, a cytokine, an humectant, a fibrous protein, an adhesion compound, a de-adhesion compound, an enzyme, or a combination thereof.

20. A cell culture article comprising:
the composition of claim 10 coated on a substrate.

21. The article of claim 20 wherein the coating further comprises a biologically active compound comprising at least one of an amino acid, a peptide, a polypeptide, a protein, a carbohydrate, a lipid, a polysaccharide, a nucleic acid, a nucleotide, a polynucleotide, a glycoprotein, a lipoprotein, a glycolipid, a glycosaminoglycan, a proteoglycan, a growth factor, a differentiation factor, a hormone, a neurotransmitter, a pheromone, a chalcone, a prostaglandin, an immunoglobin, a monokine, a cytokine, an humectant, a fibrous protein, an adhesion compound, a de-adhesion compound, an enzyme, or a combination thereof.
